(19) United States
(12) Patent Application Publication
(10) Pub. No.: US 2003/0104981 A1
(43) Pub. Date: Jun. 5, 2003

(54) Human insulin analogues

(76) Inventor: Jelena Mandic, Watertown, MA (US)

Correspondence Address: Alvin Isaacs, Esq., 9544 Hawksmoor Lane, Sarasota, FL, 34238-3221, US

(21) Appl. No.: 09/924,447

(22) Filed: Aug. 9, 2001

Related U.S. Application Data
(63) Continuation-in-part of application No. 08/552,749, filed on Nov. 3, 1995, now abandoned.

(51) Int. Cl. A61K 38/28; C07K 14/62

(52) U.S. Cl. 514/3; 530/303

(57) ABSTRACT

Novel human insulin analogues are provided for treating Diabetes Mellitus, the analogues being characterized by having enhanced stability to insulin-degrading enzyme (IDE) as well as achieving longer life times than native insulin. The insulin analogues of the invention are further characterized structurally by elimination of B26-B30 in the human insulin B-chain and by having at least one specified substitution at B10, B14 and B17.

### RELATED APPLICATION

This application is a continuation-in-part of my (previously allowed) application Ser. No. 08/552,749 filed Nov. 3, 1995.

### BACKGROUND OF THE INVENTION

This invention relates to novel insulin analogues and, more particularly, to the field of treating Diabetes mellitus by novel insulin analogues with enhanced stability towards insulin-degrading enzyme (“IDE”).

Since the starting point of the present invention can be said to be the human insulin molecule, it is appropriate to initiate this description with a description of the human insulin molecule, using the conventional abbreviations for the amino acids as stated in J. Biol. Chem. 243 (1968), 3558. The amino acids are in the L configuration.

HUMAN INSULIN MOLECULE

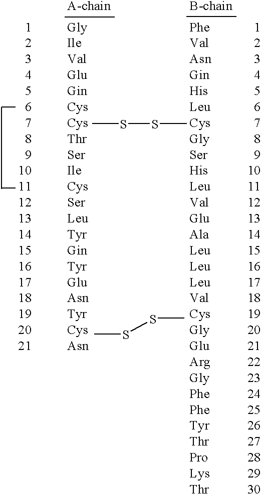

The following discussion is directed to a brief description of the history and state of the art pertaining to human insulin analogues for the treatment of humans.

Insulin analogues with extremely long half-lives may prove superior to ultralente insulin for the delivery of basal insulin. It has been proposed by W. C. Duckworth, et al., (Proc. Natl. Acad. Sci., USA, (1972) 69:3698 ) that IDE plays a key role in the cellular processing of insulin. Evidence that this proteinase is involved in the intracellular degradation of insulin includes the following:

(1) Inhibitors of the purified proteinase also inhibit insulin degradation in cells (W. C. Duckworth et al., (1981) Endocrinology, 108:1142,);

(2) the insulin cleavage sites observed in vitro with the purified proteinase are also observed in insulin extracted from cells (R. K. Assoian, et al. (1982) J. Biol. Chem., 257:9078);

(3) microinjection of monoclonal antibodies to this proteinase inhibits cellular degradation (K. Shii, et al, (1986), Diabetes, 35:675 ), and

(4) insulin can be cross-linked in intact cells to this proteinase (J. Hari, et al. (1987) J. Biol. Chem., 262:15341 ).

Diabetes mellitus is a syndrome characterized by chronic hyperglycemia and disturbances of carbohydrate, fat and protein metabolism associated with absolute or relative deficiencies in insulin secretion and/or insulin action.

Insulin, the major hormonal regulator of glucose metabolism, was first isolated from pancreatic tissue in 1921 by Frederick Banting and Charles Best. Although after its discovery, insulin was recognized to be a protein, the elucidation of its primary structure came many years later by Frederic Sanger and his coworkers. All known insulins are composed of two polypeptide chains that are linked to one another by disulfide bonds. The A- and B-chains of human, porcine and bovine insulins, like most other vertebrate insulins, are composed of 21 and 30 amino acids, respectively. These two peptide chains are covalently linked to one another by two cysteine disulfides, one between the A7-Cys and B-7Cys, and the other between the A20-Cys and B19-Cys. An additional intrachain disulfide connects cysteines A6 and A11.

The pioneering studies of the x-ray diffraction patterns of insulin crystals conducted at Oxford and the Peking Insulin Structure Group have been the cornerstone for relating insulin structure and function.

Much of what is known about insulin structure-function relationships comes from studies with modified insulins and comparisons of insulins from different animal species. An interesting recent application is the engineering of insulin analogues with altered pharmacokinetic characteristics. The different approaches are being used in attempts to provide diabetic patients with therapeutic insulins better matched to their metabolic requirements.

The first injection of insulin was given to a patient with diabetes at the Toronto General Hospital in 1922. At that time, because of the relatively short duration of action of soluble insulin, it was necessary to inject insulin subcutaneously three to four times a day to control blood glucose levels adequately. In 1936, H. C. Hagedorn discovered that the activity of insulin after injection could be delayed or prolonged with the addition of various basic proteins, e.g., fish protamine, which kept the insulin in suspension so that it was absorbed more slowly from subcutaneous sites.

In the same year, Scot and Fisher found that zinc and other heavy metals could extend the duration of action of protamine insulin even further. This finding led to the development of protamine zinc insulin (“PZI”), the first stable insulin preparation with prolonged action. An injection of PZI could lower the blood glucose for 48 to 72 and could be administered once a day by the diabetic patient. The availability of protamine insulin and PZI, which revolutionized insulin therapy, leading to the predominance of a once-a-day regimen of intermediate and long-acting insulins in the treatment of diabetes.

In subsequent years, other insulins with prolonged action were developed. Isophane insulin, a more stable form of protamine insulin also known as NPH insulin (neutral protamine Hagedorn), the most widely used insulin in the United states was introduced in 1946. Insulin zinc suspensions that contain no added protamine or modifying proteins, referred to as the “lente” (slow acting) insulin zinc suspensions, were introduced in the early 1950's.

Commercially available insulins can be classified in three categories:

### (1) Rapid-Acting Insulins

Regular insulin. So-called crystalline zinc insulin (“CZI”) permits the most rapid absorption possible after injection. When regular insulin is given subcutaneously, onset of action is delayed for 30 to 45 minutes and its total duration of action is generally considered to be approximately six hours with peak activity usually two to four hours. Regular insulin is commercially produced in phosphate-buffered and in unbuffered solution forms. The former has been found more resistant to denaturation and aggregation in the tubing administered by insulin pumps. Up to now, Regular Insulin is the only one that can be use intravenously. It is therefore used in the treatment of diabetic ketoacidosis, hyperosmolar nonketotic coma, and the maintenance of blood glucose levels during surgery, trauma, and metabolic emergencies. Most of the monomeric insulin analogues have been shown to have more rapid subcutaneous absorption profiles than regular insulin. These are expected to provide improved control of meal-related glucose levels. (Brange et al. (1990), Diabetes Care, Vol. 13, 9:923).

### (2) Intermediate-Acting Insulins.

Insulin solutions can be modified into a suspension form to delay their absorption from subcutaneous sites, thereby prolonging their action. Prolonging action of insulin can be accomplished by the addition of protamine, as in NPH insulin, or with zinc, as in the lente series of insulin. Lente insulin is a stable mixture of 30% semilente insulin and 70% ultralente insulin. NPH and Lente insulins are both commonly used, as their durations of action are similar and permit satisfactory glycemic control with one or two injections per day.

### (3) Long-Acting Insulins

The first successful protracted insulin preparation was protamine insulin, introduced in 1936 by Hagedorn and co-workers. The principle was to depress the solubility of insulin at neutral pH using a basic compound. Protamine is the generic name of strongly basic proteins present in the fish sperm cell located in nuclei in salt-like combination with nucleic acid. In theory, protamine insulin may serve as an allergen. A. B. Kurtz, et al, (Diabetologia, 16:198, (1998) ) found that the protamine-insulin complex is itself immunogenic. Also, the toxicity of protamine has been reviewed by J. C. Horrow in 1985 (Anesth Analg 64:348), In recent years, the Lente series of insulins has been used more frequently to provide diabetics' insulin requirement with one daily injection. It has been noticed that insulin preparations with protracted effect could be obtained by the addition of small amounts of zinc ions, provided that the preparations had a neutral pH and no interfering zinc-binding ions such as phosphate or citrate were present. Furthermore, the degree of protection at the same zinc concentration depended on the physical state of the suspended insulin particles, crystalline insulin particles having a longer time of action than amorphous insulin particles. This led to the development of three new protracted insulin preparations:(1) Semilente, containing only amorphous insulin particles; (2) Ultralente, containing only crystalline particles; and (3) Lente insulin, containing a 3:7 mixture of amorphous and crystalline particles insulin. Presently, ultralente insulin has been used in a variety of regimes. Its slow onset of action, long duration, and relatively small peak-of hypoglycemic effect make it useful in regimens requiring constant basal action of insulin.

### SUMMARY OF THE INVENTION

It has been reported (W. C. Duckworth, et al. (1979) Proc. Natl. Acad. Sci., USA, 76:635 ) that exposure of insulin to insulin-degrading enzyme (IDE) results in an intermediate that is composed of an intact A-chain and a B-chain cleaved between residues B16 and B17. Consequently, it has been hypothesized that an initial site of insulin cleavage by IDE was between Tyr B16 and Leu B17 residues. Tyr B16 is involved in two classes of interactions:insulin-dimer forming surface and insulin receptor binding surface (T. L. Blundell, et al., (1972), Adv. Prot. Chem., 26:279 ). The group in Geneva has prepared an engineered rat insulin analogue at B16 Tyr→Asp replacement which shows unchanged susceptibility to cleavage by IDE (I. M. Varley, et al., (1988) Eur. J. Biochem., 171:351).

Applicant has compared sequence data of the known insulins. The residues :B10, B14, B17, (taken together) are variant in guinea pig, casiragua and coypu insulins. These residues have a very interesting relation to the structure. His B10 coordinates to the zinc ions in porcine insulin hexamers. In guinea pig, this residue is asparagine, while in coypu and casiragua it is glutamine. B14 and B17, which are invariant in other insulins, all lie in the region between dimers and hexamers.

a. B14—alanine becomes threonine,

b. B17—leucine changes to serine, in casiragua, nutria, and guinea pig.

These residues are larger and more hydrophilic, and destabilize the formation of hexamers or (solubilize monomer). Furthermore, A13 leucine in guinea pig, coypu, and casiragua insulins are replaced by arginine. While A14 in guinea pig is histidine rather than Tyr, in coypu and casiragua insulin A14 is asparagine. These residues also lie in the region between dimers and would destabilize hexamers. There are no reports of rhombohedral zinc insulin crystals of guinea pig insulin. If hexamers are uniquely not required in any role of insulin in guinea pig, then these unusual modifications in sequence could be to allow the monomers to possess resistance to enzymatic degradation (in storage)?

Following this conclusion, Applicant has introduced the following modifications in the human insulin B-chain:(1) deleting B-26-B30 ; and (2) replacement of segments B10, B14, B17 and/or B25 with other amino acid substituents, which substituents are directed to the improvement of providing insulin analogues having greatly improved resistance to degradation by insulin-degrading enzyme (“IDE”), thereby, in turn, greatly increasing its life both in vitro and in vivo in the treatment of humans.

These changes will be described in detail in the following DETAILED DESCRIPTION OF THE INVENTION.

### BRIEF DESCRIPTION OF THE DRAWINGS

FIG. 1 is the amino acid sequence of the A-chain SEQ ID NO: 1 and of the B-chain SEQ ID NO: 3 showing the total amino acid sequence of the Radoyka I human insulin analogue;

FIG. 2 is the amino acid sequence of the A-chain SEQ ID NO: 1 and of the B-chain SEQ ID NO: 4, showing the total amino acid sequence of the Radoyka II human insulin analogue;

FIG. 3 shows the elution profile from DEAE-CL-6B column of crude des-(B26-B30)-[Glu-B¹⁰; Ser-B¹⁷:Tyr-B-²⁵—NH₂] Radoyka I human insulin B-chain with 0.1 M TRIS/HCl pH 7.5, gradient 250 ml of 0.5 M NaCl in 0.1 M TRIS HCl pH 7.5. The major peak was collected between 375 ml and 425 ml;

FIG. 4 shows the elution profile from DEAE-CL-6B column of crude des-(B26-B30)-[Glu-B¹⁰; Thr-B¹⁴ Ser-B¹⁷:Tyr-B²⁵—NH₂] Radoyka II human insulin B-chain with 0.1 M TRIS/HCl pH 7.5, gradient 250 ml of 0.5 M NaCl in 0.1 M TRIS/HCl pH 7.5. The peak of interest was eluted between 440 and 486 ml;

FIG. 5 shows the HPLC elution profile of recombination mixture Radoyka I insulin analogue;

FIG. 6 shows the HPLC elution profile of recombination mixture of Radoyka II insulin analogue;

FIG. 7 shows the HPLC elution profile of Radoyka II insulin analogue;

FIG. 8 shows the results of electrospray mass spectrometry of Radoyka I human insulin analogue. The work was performed on a VG MassLab Trio-2 quadruple mass spectrometer fitted with a VG BioTech Electrospray source. The samples were run at 5 μl/min in an acetonitrile:0.1% aq TFA solvent. The target compound is marked “C”;

FIG. 9 shows the results of electrospray mass spectrometry of Radoyka II human insulin analogue. The work was performed on a VG MassLab Trio-2 quadruple mass spectrometer fitted with a VG BioTech Electrospray source. The samples were run at 5 μl/min in an acetonitrile:0.1% aq TFA solvent. The target compound is marked with “E”;

FIG. 10 shows the UV absorption spectrum of Radoyka I insulin analogue;

FIG. 11 shows the UV absorption spectrum of Radoyka II insulin analogue;

FIG. 12A shows insulin receptor binding affinity of the Radoyka I and Radoyka II insulin analogues with insulin receptor in the LB lymphoma cell;

FIG. 12B shows the effect of negative cooperativity of the Radoyka I and Radoyka II insulin analogues with insulin receptor in the LB lymphoma cell;

FIG. 13 shows mitogenic effect generated by native insulin and the Radoyka I and Radoyka II insulin analogues in the LB lymphoma cell;

FIG. 14 is a graph showing the effect of NOVO insulin and the Radoyka I, Radoyka II insulin analogues on the stimulation of lipogenesis in rat adipocytes;

FIG. 15 shows the HPLC elution profile of iodinated Radoyka II insulin analogue;

FIG. 16 is a graph showing the HPLC elution profile of peak 3 (“P3”) depicted in FIG. 15;

FIG. 17 is a graph showing the HPLC elution profile of peak 5 (“P5”) depicted in FIG. 15;

FIG. 18 shows the HPLC elution profile of the degradation products of ¹²⁵I A14 porcine insulin generated after 10 minute of incubation with IDE;

FIG. 19 shows the HPLC elution profile of the degradation products of ¹²⁵I A14 porcine insulin generated after 20 minutes of incubation with IDE;

FIG. 20 shows the HPLC elution profile of the degradation products of the eluent, under peak 3 (“P3”) shown in FIG. 16, generated after 10 minutes of incubation with IDE;

FIG. 21 shows the HPLC elution profile of the degradation products of the eluent under peak 3 (“P3”) shown in FIG. 16, generated after 20 minutes of incubation with IDE;

FIG. 22 shows the HPLC elution profile of the degradation products of the eluent under peak 5 (“P5”) shown in FIG. 17, generated after 10 minutes of incubation with IDE; and

FIG. 23 shows the HPLC elution profile of the degradation products of the eluent under peak 5 (“P5”) shown in FIG. 17, generated after 20 minutes of incubation with IDE.

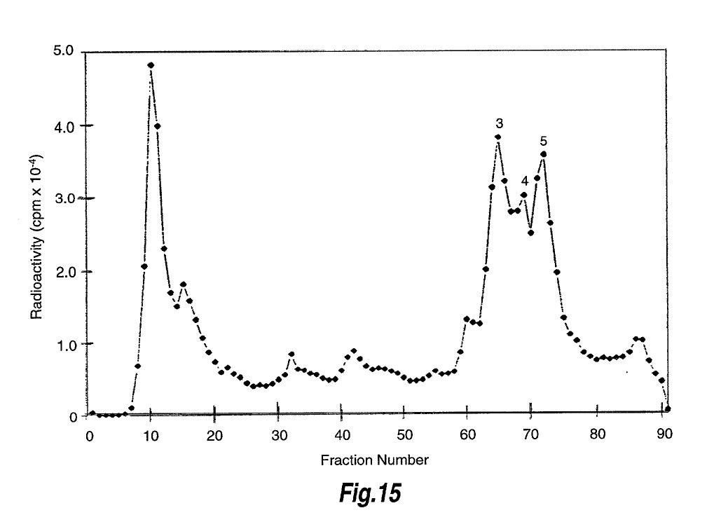

TABLE 1
TCA assay analysis of degradation of 250 μl of the radioactive
labeled derivatives (shown in FIG. 15) of Radoyka II insulin
analogue and ¹²⁵I A14 porcine insulin.

| Labeled | 10 minutes | 20 minutes |
|---|---|---|
| materials | incubation | incubation |
| ¹²⁵I A14 insulin | 25.85% | 66.79% |
| P3 | 9.7% | 16.44% |
| P4 | 15.82% | 20.42% |
| P5 | 20.75% | 25.42% |

### DETAILED DESCRIPTION OF THE INVENTION

As alluded to above, the present invention provides novel insulin analogues with higher lifetimes of duration than native insulin. The insulin analogues of the invention differ from human insulin by having at least one of the following substitutions:

1. Deletion of B26-B30 resulting in truncation of the B-chain, with substitution for the B25 phenylalanine segment of a polar amino acid containing amide substitution of acid groups (carboxamide) or unsubstituted carboxylic acid groups serves to increase resistance to IDE and, further, affects insulin receptor binding kinetics in the body, i.e., maintains high affinity of the insulin analogue to the insulin receptor on the body.

Useful polar amino acids of this description include Glycine (Gly), Serine(Ser), Threonine (Thr), Cysteine (Cys), Tyrosine (Tyr), Asparagine (Asn), Glutamine (Gln), Aspartic acid (ASP), Glutamine (Glu), Lysine (Lys), Arginine (Arg), Histidine (His), Hydroxyproline (OH-Pro), Hydroxylysine (OH-Lys), etc., Tyrosine being preferred.

2. Substitution at B14 and B17 of an amino acid that destabilizes the insulin molecule towards formation of multimers, e.g., dimers, tetramers and hexamers; and it also increases resistance to IDE.

Useful amino acids include the polar amino acids recited in 1., immediately above, with Thr and Ser being preferred.

3. Substitution for Histidine at B10 of an amino acid that increases resistance to cleavage by IDE and that affects insulin binding kinetics to insulin receptors in the body.

Useful amino acids include Glutamic Acid (Glu), Glutamine (Gln), and Asparagine (Asn), with Glutamic Acid being preferred.

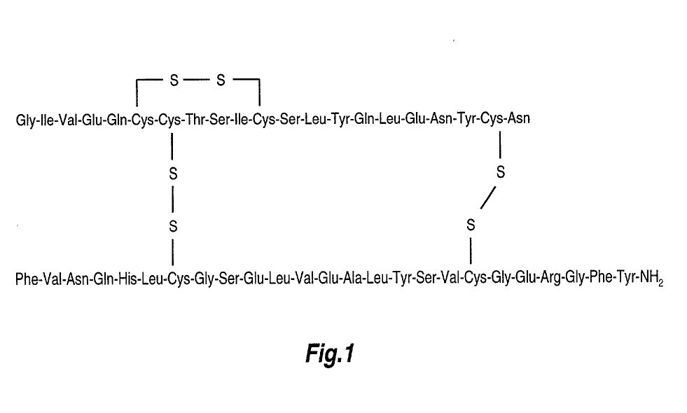

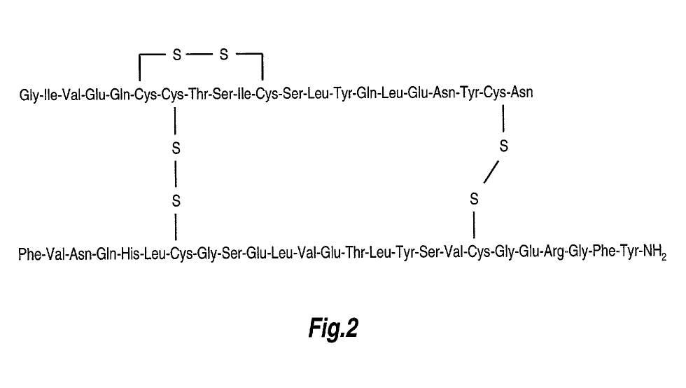

As further illustrations of the present invention, attention is invited to FIGS. 1 and 2. These novel analogues of human insulin have been chemically prepared with the following arrangements:

1. des-(B26-B30)-[Glu-B¹⁰; Ser-B¹⁷:Tyr-B²⁵—NH₂] insulin analogue having the code name selected by Applicant:Radoyka I human insulin analogue having A-chain SEQ ID NO. 1 and B-chain SEQ ID NO. 3; and

2. des-(B26-30)-[GluB¹⁰:Thr-B¹⁴; Ser-B¹⁷:TyrB²⁵—NH₂ insulin analogue (code name:Radoyka II human insulin analogue having A-chain SEQ ID NO: 1 and B-chain SEQ ID NO: 4

Comparative studies of live-time of native insulin analogues by IDE were investigated. The Radoyka II insulin analogue was shown to possess 4.5 times higher stability from insulin-degrading enzyme than that of ¹²⁵IA14 porcine insulin, as judged by trichloroacetic acid (“TCA”) precipitation or high performance liquid chromatography (“HPLC”) assay. The other insulin analogue, Radoyka I, is believed to contain approximately the same resistance to degradation by IDE. It is also believed that the hydrophobic character of the backbone at B14 and B17 which was replaced by hydrophilic amino acids is contributing factor to the five times higher performance displayed by this analogue. The substitution of glutamic acid for histidine at position 10 of the B-chain and/or the elimination of the B26-B30 segment with substitution of the B25 with Tyr-α-carboxamide was chosen to maintain high affinity of these analogues to insulin receptor. Also, appropriate substitution at B10 and B25 can be a factor contributing to higher life-time of the insulin molecule. B10 was found to be one of the cleavage points by IDE, while Phe B25 was involved in formation of insulin-insulin degrading enzyme complex.

It has been reported (W. C. Duckworth, et al. (1979) Proc. Natl. Acad. Sci., USA, 76:635.) that exposure of insulin to insulin-degrading enzyme results in an intermediate that is composed of intact A-chain and B-chain cleaved between residues B16 and B17. Consequently, it has been hypothesized that an initial site of insulin cleavage by IDE was between Tyr B16 and Leu B17 residues. Tyr B16 is involved in two classes of interactions:insulin-dimer and insulin-receptor binding surface (T. L. Blundell, et al., (1972), Adv. Prot. Chem., 26:279 ). The group in Geneva has prepared an engineered rat insulin analogue at B16 Tyr→Asp replacement which shows unchanged susceptibility to cleavage by IDE (I. M. Varley, et al., (1988) Eur. J. Biochem., 171:351).

Applicant has compared sequence data of the known insulins. The residues:B10, B14, B17, (taken together) are variant in guinea pig, casiragua and coypu insulins. These residues have a very interesting relation to the structure. His B10 coordinates to the zinc ions in porcine insulin hexamers. In guinea pig, this residue is asparagine, while in coypu and casiragua it is glutamine. B14 and B17, which are invariant in other insulins, all lie in the region between dimers and hexamers.

a. B14—alanine becomes threonine

b. B17—leucine changes to serine, in casiragua, nutria, and guinea pig.

These residues;are larger and more hydrophilic, and destabilize the formation of hexamers or solubilized monomer. Furthermore, A13 leucine in guinea pig, coypu, and casiragua insulins are replaced by arginine. While A14 in guinea pig is histidine rather than Tyr, in coypu and casiragua insulin A14 is asparagine. These residues also lie in the region between dimers and would destabilize hexamers.

There are no reports of rhombohedral zinc insulin crystals of guinea pig insulin. If hexamers are uniquely not required in any role of insulin in guinea pig, then these unusual modifications in sequence could be to allow the monomers to possess resistance to enzymatic degradation in storage.

Following this conclusion, Applicant has introduced the above-mentioned modifications in human insulin for increasing the affinity towards the insulin receptor in the body while at the same time materially decreasing catalytic activity towards insulin-degrading enzyme (IDE).

It is these modifications and the substantially improved results obtained thereby upon which patentable novelty is herein predicated!

These novel human insulin analogues, as exemplified by the aforementioned analogues identified as Radoyka I and Radoyka II, respectively, can be produced by any of a variety of techniques known to those skilled in the art.

For instance, the A- and B-chains of Radoyka I and Radoyka II insulin analogues can be synthesized by any of the known peptide synthesis techniques, including solid phase peptide synthesis as well as solution techniques, e.g., fragment condensation. The insulin analogues of the invention can also be prepared by combining of porcine A-chain, which is identical with the respective chain of human insulin, with des- (B-26-B30)-[Glu-B¹⁰; Ser-B¹⁷:Tyr-B²⁵—NH₂] code name Radoyka I human insulin B-chain SEQ ID NO: 3) and des-(B26-B30)-[Glu-B¹⁰; Thr-B¹⁴; Ser-B¹⁷:Tyr-B²⁵—NH₂] code name (Radoyka II human insulin B-chain SEQ ID NO: 4 ) thereof prepared by peptide synthetic techniques or recombinant DNA methods. Also, other A-chain analogues could be combined with the altered B-chains of this invention.

In general, to prepare Radoyka I and Radoyka II human insulin analogues requires human or porcine insulin A-chain, obtained by any known technique, be combined with Radoyka I human insulin B-chain or Radoyka II human insulin B-chain prepared by any convenient technique.

The A-, Radoyka I B-, Radoyka II B-chains of human insulin are preferably in their stabilized S-sulfonate forms which can be recombined by known procedures to form the intact active insulin analogues of the invention. Once the recombination reaction has been completed, the Radoyka I and Radoyka II insulin analogues can be isolated and assayed for purity and activity by a variety of techniques known to those skilled in the art. Commonly employed techniques for purification of insulin and insulin analogues are chromatographic techniques, such as high performance liquid chromatography (“HPLC”), gel filtration and ion exchange chromatography. Purity of product can be determined by a variety of techniques including HPLC, polyacrylamide gel electrophoresis, amino acid analysis, amino acid sequencing and mass spectrometry.

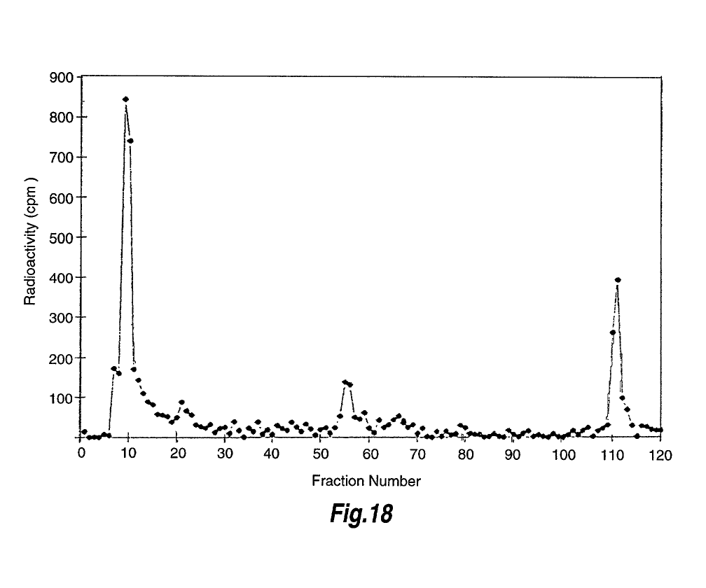

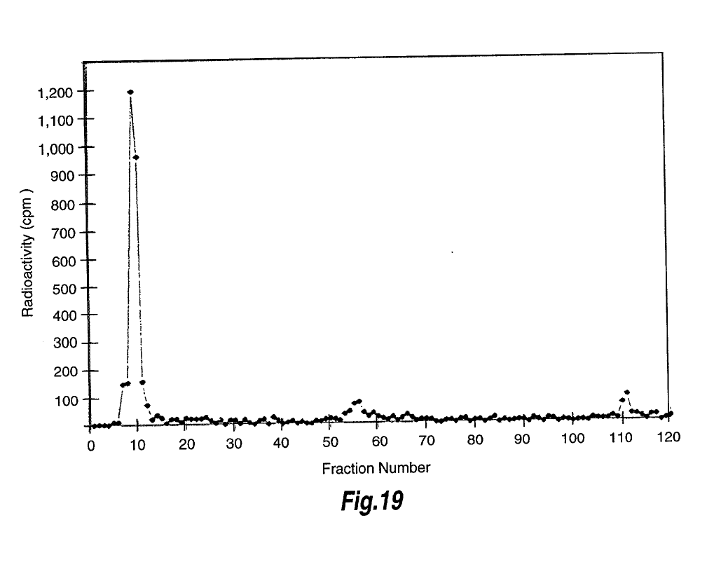

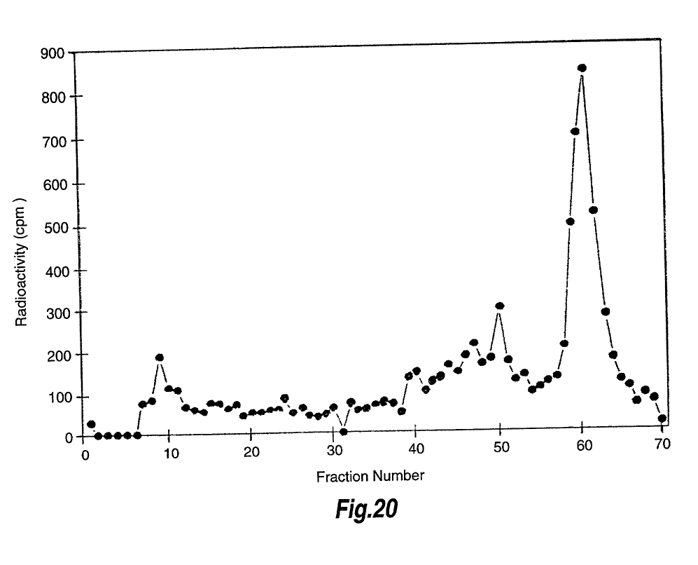

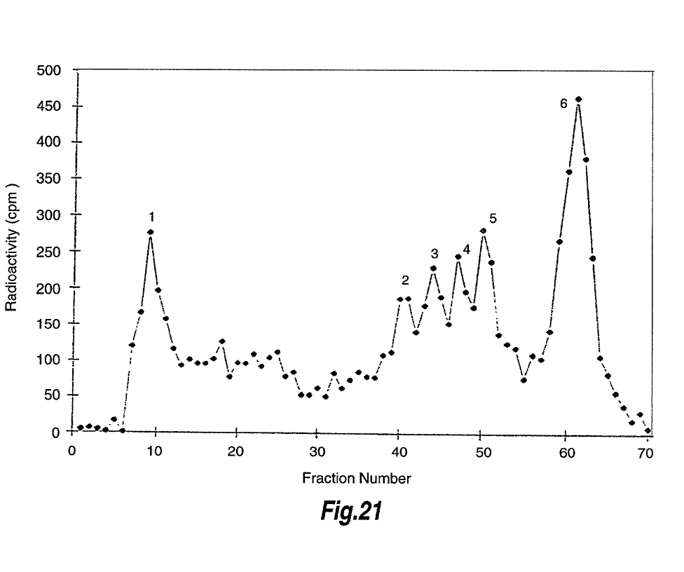

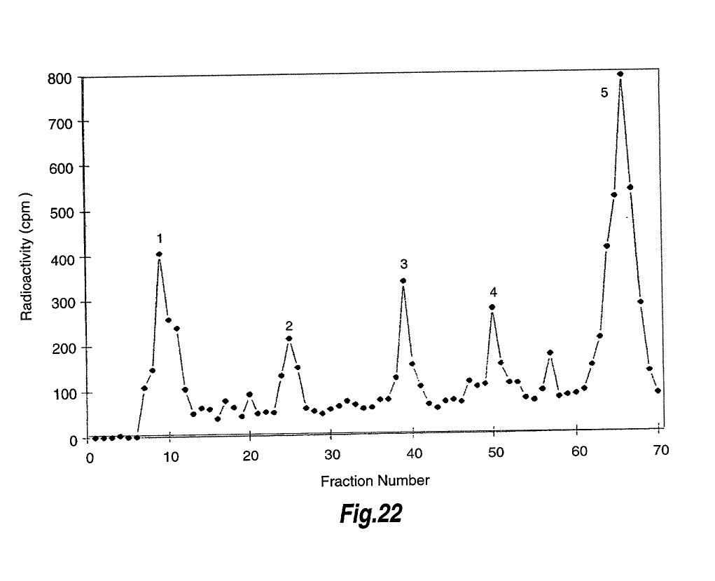

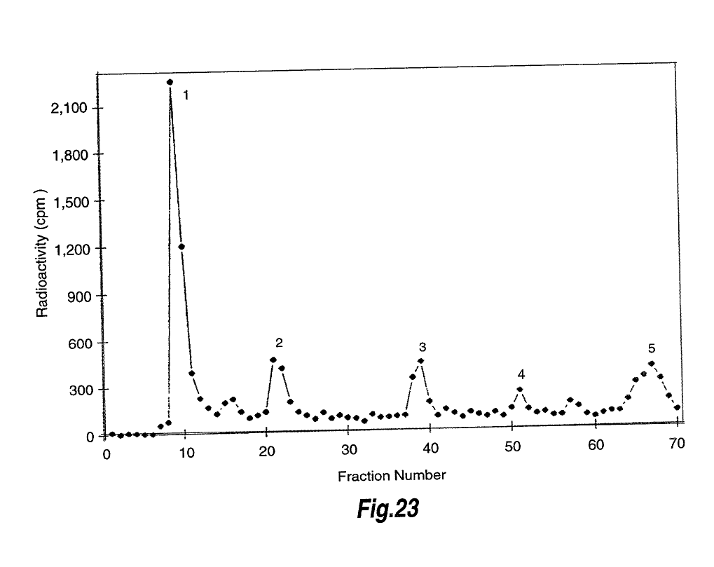

The Radoyka I and Radoyka II insulin analogues may be formulated into pharmaceutical compositions for administration to diabetic patients, The pharmaceutical compositions comprise the insulin analogues of the invention in an amount which is therapeutically effective in promoting the attainment of hormonal homeostasis in diabetic patients with action of duration of at least 24 hours. Therefore, the Radoyka I and Radoyka II human insulin analogues might provide a twice-weekly or once-a-week regimen for administrating to diabetic patients. Most importantly, patients with the syndrome of subcutaneous insulin resistance, as a result of increased activity of insulin-degrading enzyme (“IDE”), E. Paulson et al., (1979) Diabetes, 28:640) can be successfully treated by Radoyka I plus/or Radoyka II insulin analogue(s) mixed with a pharmaceutically acceptable carrier. The insulin analogues of the invention have been found to be more resistant to enzymatic degradation by IDE (see FIGS. 20, 21, 22 and 23) than native insulin (see FIGS. 18 and 19). The TCA assay results are shown in Table 1. Also, since Radoyka I and II insulin analogues have been shown to be the slowest analogues which dissociate from insulin receptor, they can be a promising solution for treating patients with Mendenhall's syndrome, congenital disorder characterized by short stature, somatic abnormalities, and severe insulin-resistant diabetes mellitus. Severe insulin resistance is due to decreased binding of the insulin molecule to its membrane receptor. Most patients with this syndrome have died in childhood from ketoacidotic coma. Currently, Mendenhall's syndrome has been treated by insulin-like growth factor I (“IGF-I”), (J. D. Quin, et al., (1990), The New England Journal Of Medicine, Vol. 323, 20:1425).

The invention is further illustrated by the following specific examples which are not intended in any way to limit the scope of the invention.

### EXAMPLE 1

### The Synthesis of two Human Insulin Analogues by Solid Phase Method

DES-(B26-B30)-[Glu-B¹⁰; Ser-B¹⁷; Tyr-B-²⁵—NH₂] RADOYKA I HUMAN INSULIN (having A-chain SEQ ID NO: 1 and B-chain SEQ ID NO: 3); and DES-(B26-B30) [Glu-B¹⁰; Thr-B¹⁴; Ser-B¹⁷; Tyr-B²⁵—NH₂] RADOYKA II HUMAN INSULIN (having A-chain SEQ ID NO: 1 and B-chain SEQ ID NO: 4)

The Synthesis of Radoyka I and Radoyka II human Insulin Analogues from Porcine A-Insulin Chain and the Synthetic Radoyka I SEQ ID NO: 3 and RADOYKA II SEQ ID NO: 4 Human B-Insulin Chains.

The present work was started with the object of cleaving the disulfide bonds of porcine insulin by a method which would give the two chains as stable derivatives, readily separable from each other and from unmodified insulin. The treatment of insulin with sodium sulfite in the presence of a mild oxidizing agent (oxidative sulfitolysis) causes its disulfide bonds to be broken and the A- and B-chains are converted to the corresponding S-sulfonated derivatives (J. M. Swan) (1957) Nature, 180:643; L. Bailey et al., (1959) J. Boil

### Synthesis of des-[B26-B30](Glu-B10; Ser-B17; Tyr-B25-NH₂) Radoyka I Human Insulin B-Chain SEQ ID NO: 3 and des-[B26-B30](Glu-B¹⁰; Thr-B¹⁴:Ser-B¹⁷; Tyr-B²⁵—NH₂) Radoyka II Human Insulin B-Chain SEQ ID NO: 4

The S-sulfonated Radoyka I and II human insulin B-chains were assembled by stepwise solid-phase synthesis (R. B. Merrifield (1963) J. Am. Chem. Soc., 85:2149 ; G. Barany, et al., (1980) In:The Peptides, E. Gross and J. Meienhofer, eds (Academic, New York) Vol. 2) by using 2 g of 4-methybenzhydrylamine resin (“MBHA”) as the solid support (0.43 mmol/g).

The Protected Amino Acids

The t-butyloxycarbonyl group was used for N-α protection except for the N-terminal phenylalanine residue, which was protected by a benzyloxycarbonyl group.

The Side-Chain Protecting Groups Were as Follows:

-benzyl for serine

-2,6-dichlorobenzyl for tyrosine

—N^{G}p-toluene sulfonyl (TOS) for arginine

-benzyloxymethyl (BOM) for histidine

-4-methylbenzyl for cysteine

-cyclohexyl for glutamic or aspartic acid

The peptides were synthesized by using a shaker for manually operated solid-phase peptide synthesis designed by Merrifield in 1963. A manual coupling protocol was followed (R. B. Merrifield, et al., (1982) Biochemistry, 21:5020 with activated protected amino acids, 1-hydroxybenzotriazole (“HOBT”). Dicyclohexylcarbodiimide (“DCC”) in 25 ml of dimethylformamide (“DMF”). 3-fold excess of coupling components were applied to the resin and the completion of reaction was monitored by getting of negative ninhydrin test (E. Kaiser, et al., (1970), Anal. Biochem., 34:595). B25-B15 fragment bound resin was split into two equal portions. In one reaction vessel after the deprotection step, threonine was coupled at the B14 position; while the other was coupled at the usual alanine amino residue at B14. After the chains were totally assembled, the peptide-resins were extensively washed with methylene chloride, methanol and dried (weight:3.0g). The cleavage and deprotection of B-chains from the resin was performed by the low/high hydrogen fluoride procedure (J. P. Tam et al,(1983) J. Am. Chem. Soc., 105:6442). 500 mg of the peptide-resin amount was in the first step treated with a mixture consisting of 1.0 ml of p-cresol, 6.5 ml of dimethyl sulfide and 2.5 ml of hydrogen fluoride. The reaction mixture was left for 2 hours at 0° C. (low HF procedure). In the next step, the reaction mixture was concentrated under reduced pressure and the residue was treated with 1.0 ml of p-cresol and 9.0 ml of hydrogen fluoride for about 1 hour at 0° C. (high HF procedure). The residue was washed with ethyl acetate in 3×50 ml portions. To the suspension of this product in 25.0 ml of 8M guanidine hydrochloride buffered with 0.1M TRIS HCl, pH 8.8, 600.0 mg of sodium sulfite and 300.0 mg of the sodium tetrathionate were added. After 1.5 hours, 600.0 mg of sodium sulfite and 300.0 mg of sodium tetrathionate were added to the reaction mixture. The total time of reaction was 3 to 3.5 hours. The reaction mixture was filtered to remove the resin, and the filtrate was placed in spectra/POR membrane tubing # 3 and dialyzed against four changes of distilled water (4.0 l each) at 4° C. for 24 hours. Lyophilization of dialysate afforded the crude S-sulfonated B-chains.

Yields:

1. des-[B26-B30](Glu-B¹⁰; Ser-B¹⁷; Tyr-B²⁵—NH₂) Radoyka I human insulin B-chain SEQ ID NO: 3, 100.95 mg.

2. des-[B26-B30](Glu-B¹⁰; Thr-B¹⁴; Ser-B¹⁷; Tyr-B²⁵—NH₂ ) Radoyka II human insulin B-chain SEQ ID NO: 4 131.0 mg.

51.7 mg of crude Radoyka II human insulin B-chain SEQ ID NO: 4 were dissolved in 3 ml of 0.1M TRIS/HCl buffer pH 7.5. The solution was centrifuged and the pellet was washed three times with the same buffer. The collected supernatants were placed to DEAE-CL-6B (2×23 ml). The peak of interest was eluted between 440 and 486 ml of effluent. The effluents were dialyzed against distilled water with Spectra/POR membrane tubing # 3 and lyophilized. From the 51.7 mg of crude material, 17.0 mg of product were obtained as a white fluffy material (FIG. 4). 55.3 mg. of crude Radoyka I human insulin B-chain SEQ ID NO: 3 were dissolved in 4 ml of 0.1M TRIS/HCl pH 7.5.

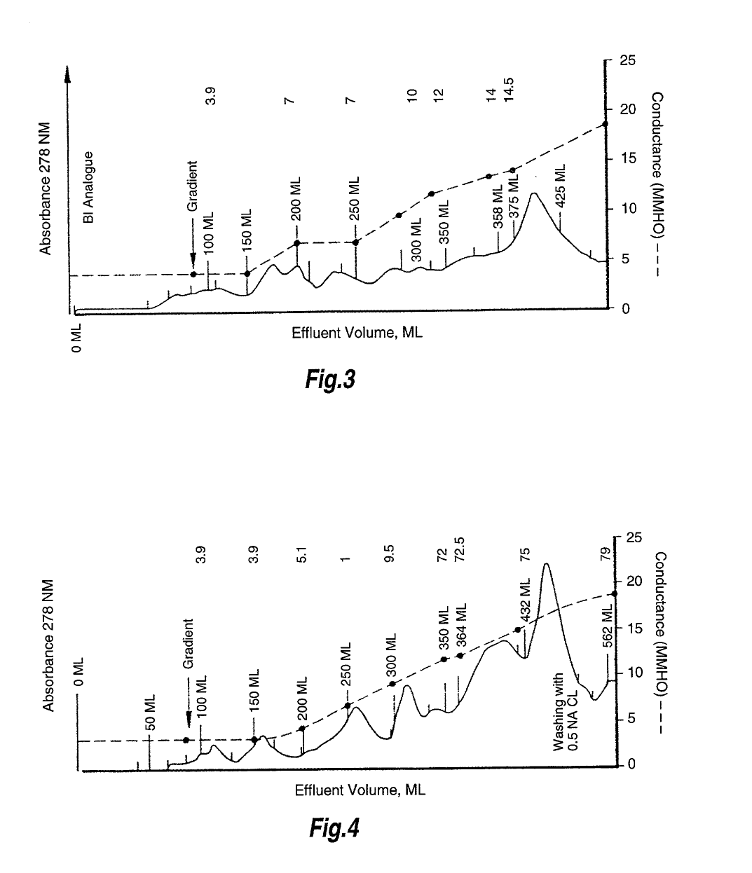

The same protocol was used as with the previous synthetic B-chain. The major peak was collected, and it was eluted between 375 and 425 ml of effluent. The effluent under the main peak (FIG. 3) was dialysed against distilled water (spectra/POR membrane tubing # 3) and lyophilized. 17.8 mg of purified product were obtained as s white fluffy powder.

### Synthesis of: des-[B-26-B30](Glu-B¹⁰; Ser-B¹⁷; Tyr-B²⁵—N:H₂) Designated Radoyka I Human Insulin Analogue having A-chain SEQ ID NO: 1 and B-Chain SEQ ID NO: 3; and des [B26-B30](Glu-B¹⁰; Thr-B¹⁴ Ser-B¹⁷:Tyr-B²⁵—NH₂) Designated Radoyka II Human Insulin Analogue having A-Chain SEQ ID NO: 1 and B-Chain SEQ ID NO: 4)

The S-sulfonated human A-chain SEQ ID NO: 1, which is identical with the respective chain of porcine insulin (D. S. H. W. Nicol, et al., (1960) Nature, 181:483) was prepared by oxidative sulfitolysis of porcine insulin and separation of the resulting S-sulfonated A- and B-chains by CM-cellulose chromatography (P. G. Katsoyannis, et al. (1967), Biochemistry, 6:2635 ), the only difference being that sulfitolysis was performed for 3.5 hours instead of for 24 hours.

Two recombination mixtures were performed for the synthesis of the desire human insulin analogues.

1. 10.25 mg of S-sulfonated Radoyka I human insulin B-chain SEQ ID NO: 3,

20 mg of S-sulfonated porcine (identical with human) A-chain,

4.4 mg of dithiothreitol DTT,

5.0 ml of 0.1M pH 10.5 glycine buffer

2. 10 mg of S-sulfonated Radoyka II human insulin B-Chain SEQ ID NO: 4,

20.00 mg of S-sulfonated porcine (identical with human) A-chain,

4.4 mg of dithiothreitol,

5.0 ml of 0.1M glycine buffer pH 10.5

After the 24th hour at 4° C., the mixtures were diluted with glacial acetic acid (1.0 ml), and the resulting precipitates were removed by centrifugation for five minutes (International HN; 3000 rpm). The supernatants, containing the active material, were passed through a 0.45 μm cellulose acetate filter (Sartorius).

Amino acid analysis of the synthetic des-[B26-B30](Glu-B¹⁰; Ser-B¹⁷ ; Tyr-B²⁵—NH₂) Radoyka I human insulin B-chain SEQ ID NO: 3 after acid hydrolysis gave the following composition, expressed in molar ratios:Asp 0.93(1); Glu 3.93 (4); Ser 1.83 (2); Gly 2.96 (3); Ala 1.12 (1); Al 2.89 (3); Leu 3.02 (3); Tyr 2.08 (2); Phe 1.68 (2); 1.73 (1); Arg 1.39 (1); (cysteine was not determined).

Amino acid analysis of the synthetic des-[B26-B30](Glu-B¹⁰; Thr-B¹⁴; Ser-B¹⁷; Ser-B¹⁷; Tyr-B²⁵—NH₂) Radoyka II human insulin B-chain SEQ ID NO: 4 after acid hydrolysis gave the following composition expressed in molar ratios:Asp 0.97 (1); Thr 1 (1); Ser 1.77 (2); Glu 4,29 (4); Gly 3.14 (3); Val 2.79 (3), Leu 3.00 (3) Tyr !0.91 (2); Phe 1.60 (2); His 1.02 (1); Arg 1.21 (1) (cysteine was not determined).

### EXAMPLE 2

After being synthesized, the human insulin analogues of the invention have been purified by reversed phase HPLC, using 0.2 M pH 4 ammonium acetate buffer and acetonitrile as eluents.

The Purification of Radoyka I and Radoyka II Human Insulin Analogues

The HPLC separations were performed with a Varian model 5000 liquid chromatograph fitted with a 1000-μl loop. The column used was a Brownlie aquapore C-18 wide pore (300 A) (7.5 mm×250 mm). The insulin analogues were eluted from the column with 0.2M ammonium acetate (pH 4.0) acetonitrile solvent system.

| | |
|---|---|
| Solvent A: | 85% 0.2 M pH 4 Ammonium acetate 15% acetonitrile |
| Solvent B: | 60% 0.2 M pH 4 ammonium acetate; 40% acetonitrile |

Flow was maintained at 1.0% ml/min throughout the elution. The isocratic and linear gradient steps in acetonitrile concentration are described under “Mandic Method”.

1) 5min at 15%

2) 15 min gradient to 23.75%

3) 20 min gradient at 23.75%

4) 10 min gradient at 40 %

5) 5 min gradient at 40%

6) 5 min gradient to 15%.

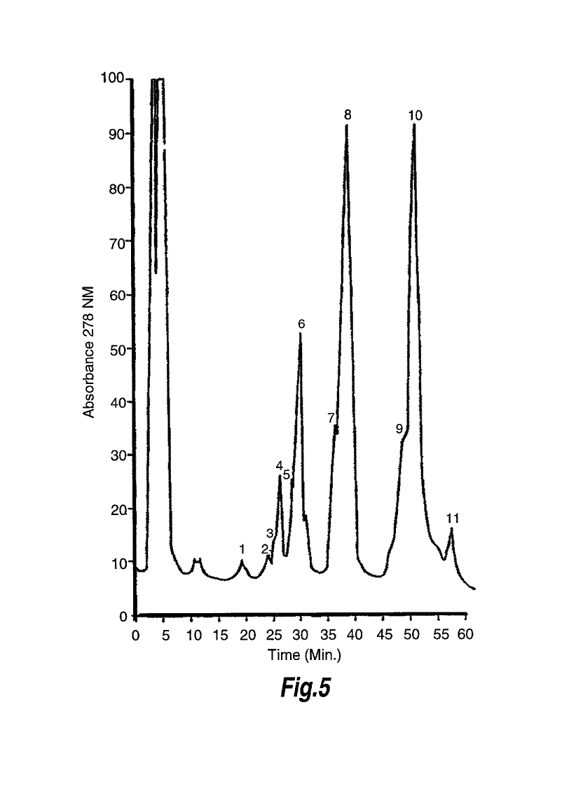

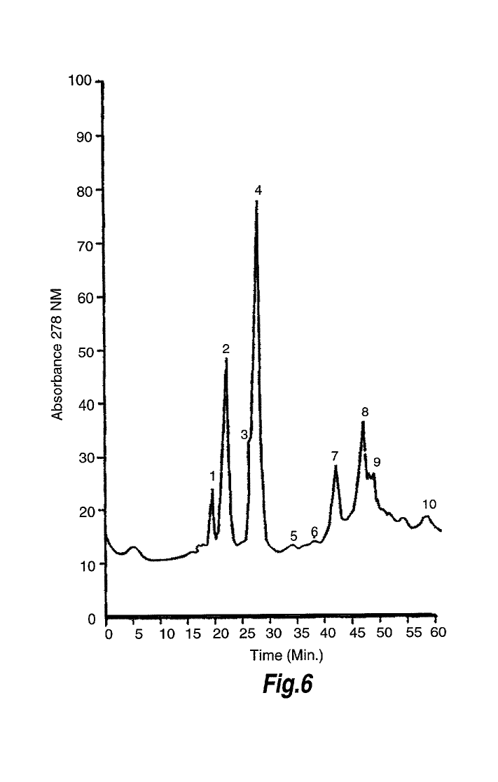

FIG. 5 and FIG. 6 show the HPLC elution profiles of recombination mixture of Radoyka I and Radoyka II human insulin analogues.

### EXAMPLE 3

Each of the peaks seen in FIGS. 5 and 6 was collected and lyophilized. The lyophilized material was subjected to electrospray mass spectrometry using a VG MassLab Trio-2 quadrupole mass spectrometer fitted with a VG BioTech Electrospray source. The samples were run at 5 μ/min in acetonitrile:0.1% TFA in water. The target compounds were present in peaks numbered 10 and 8 of their respective chromatographs FIGS. 5 and 6. The ions at 5197.0 and 5227.8 correspond to the molecular mass of Radoyka I and Radoyka II human insulin analogue. The elution profiles FIGS. 5 and 6 from HPLC of chain recombination reactions are, as expected, fairly complex. Although the excess A-chain method shifts the otherwise statistical mix of recombination products towards more intact insulin, significant amounts of other species are generated. Peaks 8 and 10 appeared to be good candidates for intact product as they eluted closest to native insulin in this gradient system. Electrospray mass spectrometry confirmed that these peaks did indeed contain the required product but also other species. The Radoyka I analogue, for instance, showed two extra species (FIG. 8) whose masses correspond to intact B-chain (2817 Da) and A-chain dimer (4759 Da). The Radoyka II analogue showed four extra species which correspond in mass to B-chain (2847), B-chain-cysteic acid (2946), A-chain dimer (4759) and A-chain dimer one residual S-sulfonate (4840) (FIG. 9).

### EXAMPLE 4

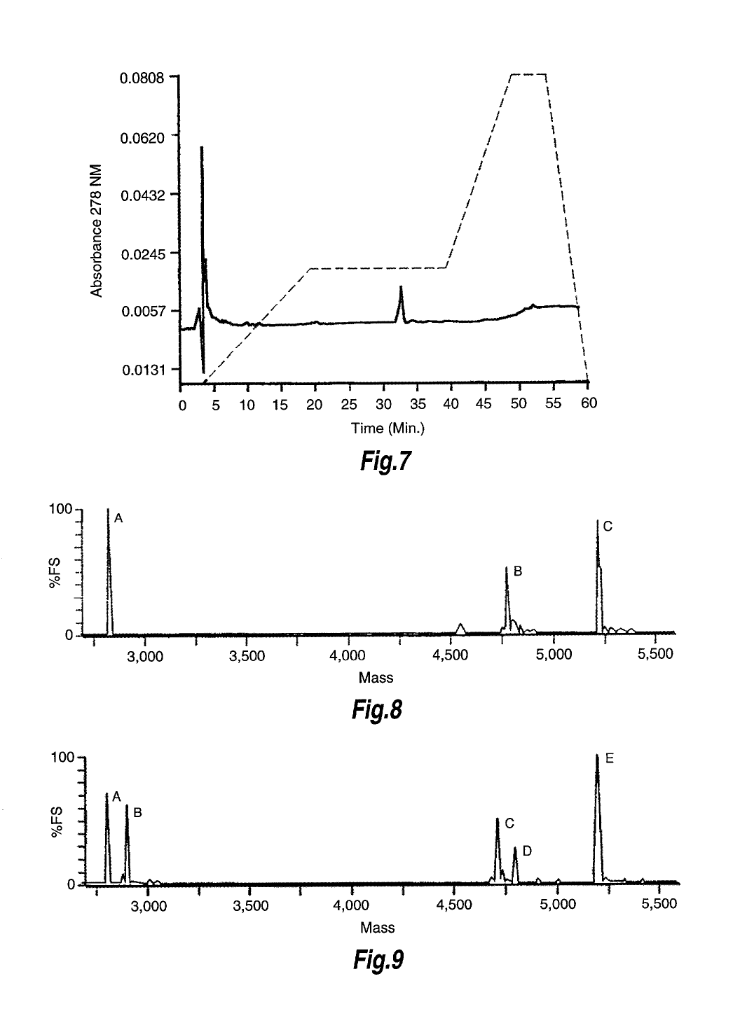

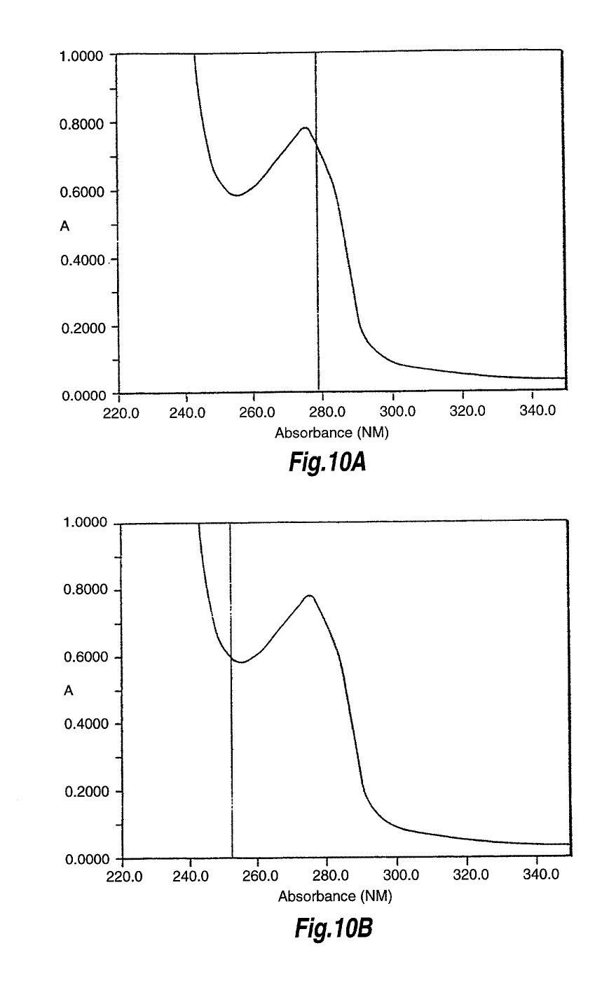

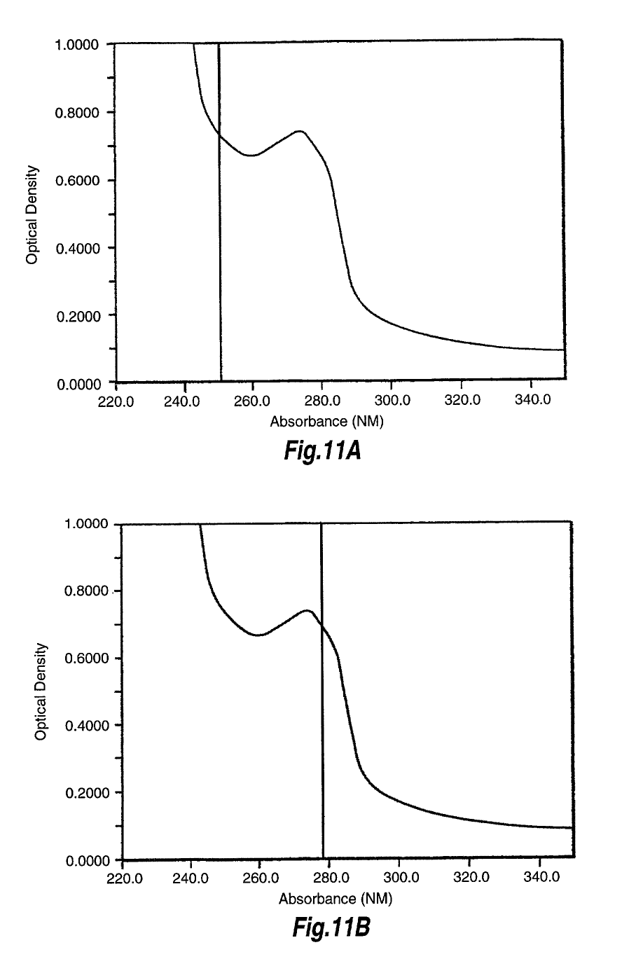

The optical density of Radoyka I and Radoyka II human insulin analogue was determined by spectrophotometer (Elmer Perkin UV/VIS Spectrometer Lambda 2) (FIGS. 10A,B and 11A,B. Extinction coefficient of Radoyka I human insulin analogue is 1.001923 and of Radoyka II human insulin analogue 0.994454, yields are 1.8 mg and 2.2 mg, respectively, from 1,5 mixture proportion of the A- and B-chains described under the synthesis method. Yields based on B-chain are usually about 10%. In the case of Radoyka I insulin analogue it was 4.09% and Radoyka II insulin analogue it was 4.98%. In reversed-phase HPLC, des-(B26-B30)-[Glu-B¹⁰; Thr-B¹⁴; Ser-B¹⁷; TyrB²⁵—NH₂] human insulin analogue was eluted significantly earlier (FIG. 7) than was native insulin being eluted at 51 minutes. This behavior indicates that the synthetic analogue is a more polar molecule.

### EXAMPLE 5

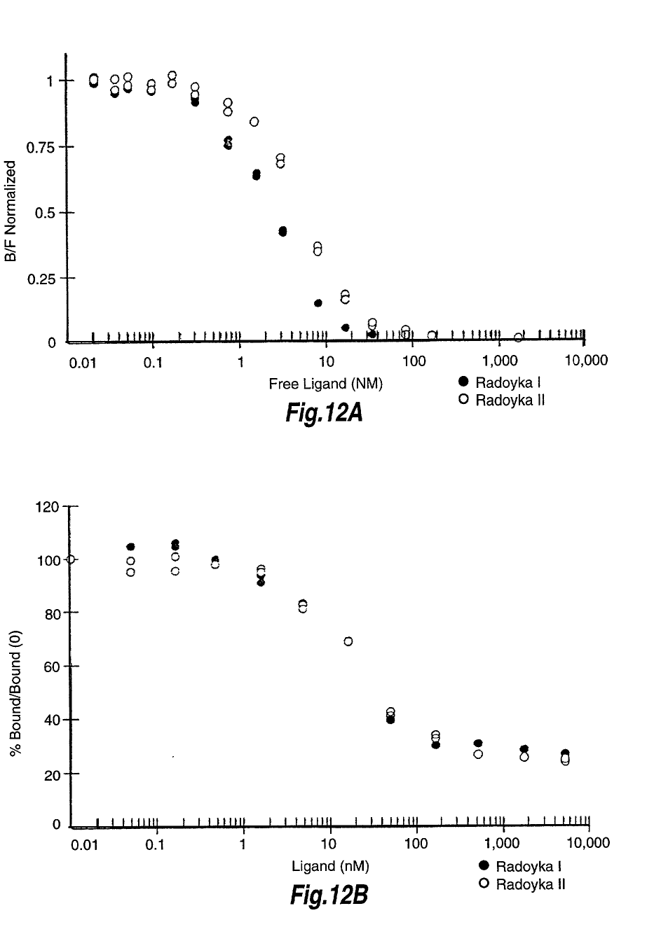

Insulin binding, association kinetics, pH dependence and dissociation kinetics were assayed to LB T cell lymphoma line, which is entirely devoid of IGF-I receptors. Competition of Radoyka I and Radoyka II human insulin analogues with native insulin for insulin receptors in the LB lymphoma cell were measured. In this assay, Radoyka I and Radoyka II human insulin analogues displayed 5 times. and 10 times less binding affinity than native insulin, respectively. This corresponds to 20% of Radoyka I and 10% Radoyka II that of native insulin. The results of the assay are shown in FIG. 12A. The study of association and dissociation kinetics of Radoyka I and Radoyka II human insulin analogues for the purified ectodomain of insulin receptors were performed by BIACORE Pharmacia instrument. Association and Dissociation constants were:

Kₐ=S⁻M⁻¹(×10⁴) for

Radoyka I human insulin analogue 0.93

Radoyka II human insulin analogue 0.73

human insulin 3.23

Radoyka II human insulin analogue has the lowest association constant of any yet described.

K_{d}=S^{-M-1}(×10³) for

Radoyka I human insulin analogue 0.82

Radoyka II human insulin analogue 0.53

human insulin 3.46

Radoyka II human insulin analogue has the slowest dissociation from the insulin receptor of any dissociatable analogue yet reported.

The Radoyka I and Radoyka II insulin analogues do not show disappearance of negative cooperativity about 0.1 μM and displayed a monophasic instead of a bell-shaped curve (plotting the ratio of bound to free ligand). It has been shown by P. De Meyts in press) that the key residues determining the loss of negative cooperativity at high insulin concentration, are A13 Leu and B17 Leu. Radoyka I and Radoyka II human insulin analogues possess at B17, Ser residue. Also, they were showed to be less negatively cooperative than unlabeled insulin. The results are shown in FIG. 12B.

Biological potency of Radoyka I and Radoyka II human insulin analogues (lipogenesis in rat adipocytes) were 77% and 22% respectively, compared with negative insulin, as shown in FIG. 14.

Interestingly, the substitution at B¹⁴Ala→Thr in Radoyka II insulin analogue reduced potency 3.5 times in comparison with Radoyka I insulin analogue. The residues B¹⁴Ala have not previously been reported as important in metabolic signaling. It has been assumed that des-(B26-B30)-[Glu-B¹⁰; Tyr-B²⁵—NH₂] insulin analogue is at least 20 times more active than native insulin. If so, Ala B¹⁴→Thr and Leu B¹⁷→Ser reduced the biological potency of des-(B26-B30)-[Glu-B¹⁰, Tyr-B²⁵—NH₂] insulin analogue at least 19 times. With this, it can be established that Ala B¹⁴ and Leu B¹⁷ are involved in metabolic signaling.

The action in vivo of Radoyka I and Radoyka II insulin analogues can be envisaged leading to some therapeutic advantages if the slow kinetics lead to an altered distribution of the analogue after peripheral administration.

It has been shown that in the lymphoma cell line, which is entirely devoid of IGF-1 receptors, that the mitogenic potency of a variety of insulin analogues is inversely related to their dissociation rate from insulin receptor (P. De Meyts, et al., (1993), Exp. Clin. Endocrinal, Leipzig,.101:102).

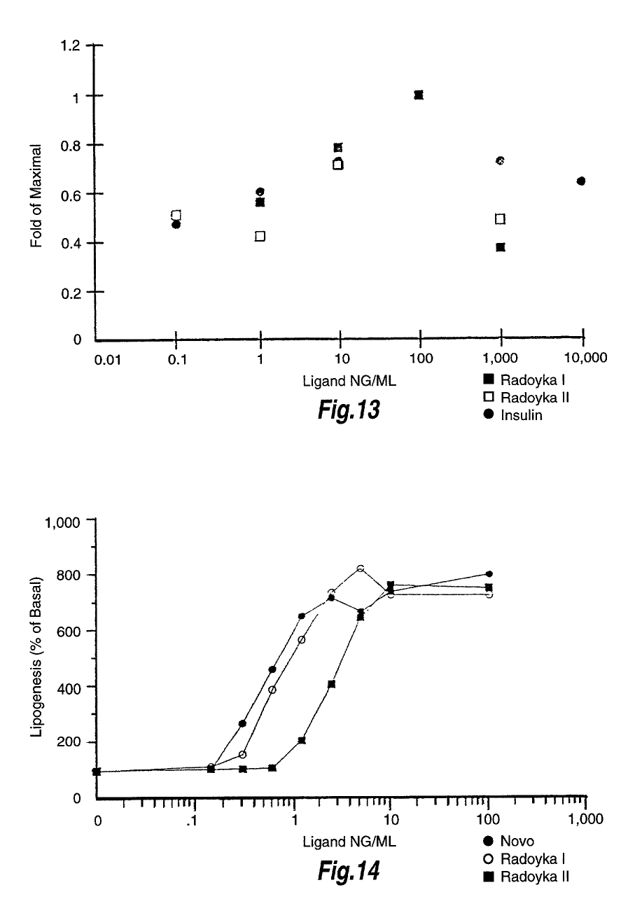

The most potent mitogen A8 His B10 Glu B27 His (code name H2) insulin bound essentially irreversibly to LB and IM-9 (K. Drejar (1992), Diabetis/Metabolism Reviews, 8:259. The Radoyka I and especially Radoyka II insulin analogue (the slowest insulin analogue) showed the slowest dissociation rate from insulin receptors. They displayed almost the same mitogenic potency as native insulin (FIG. 13). From above-mentioned des-(B26-B30)-[Glu-B¹⁰; Thr-B¹⁴; Ser-B¹⁷; Tyr-B²⁵—NH₂] insulin analogue (code name Radoyka II human insulin analogue) should be one of the most mitogenic analogues yet described.

Also, des-(B26-B-30(-Glu-B¹⁰; Ser-B¹⁷; Tyr-B²⁵—NH²] insulin analogue (code name Radoyka I human insulin analogue) should be the second order in a scale of mitogenic potency, Radoyka I displayed the slower dissociation rate than H2.

The above data show that there is no simple relationship between the dissociation rate and mitogenic signaling (stimulation of thymidine incorporation) of insulin or insulin analogues. It is proposed that the duration of the half-life of the insulin receptor complex does not regulate fundamental mechanisms to membrane signaling such as metabolic or mitogenic effects. Radoyka II and Radoyka I analogues de-couple the kinetic behavior, i.e. the dissociation and association rate from the biological effects such as mitogenic and metabolic signaling. It is believed that “biological signaling” at least in vitro is rather a matter of chemical and structural behavior of the ligand-receptor complex. It was found that B10 His replaced by Asp in native insulin showed a significantly increased mitogenic effect (poster presentations at American Diabetes Association, “Carcinogenic Effect on Female Rats after 12 months Administration of the Insulin Analogue B10 Asp”,. San Antonio, Tex., 1992). Of particular interest would be to investigate B10 Glu insulin in relation to mitogenic signaling. It is expected that the mitogenicity of B10 Glu insulin to be higher than both native insulin and B10 Asp insulin. The most potent insulin analogue H2 includes at B10 Glu amino residue. If so, then Radoyka I insulin analogue with the substitutions at B17 Ser ; B25 Tyr-NH₂ and des-B26-B30 may somehow cancel the mitogenic signaling of B10 Glu residue and level up with native insulin. Radoyka II insulin analogues possess the additional substitution at B14 Thr; which causes a lower mitogenic effect in comparison with insulin and Radoyka I insulin analogue. From above, it could be proposed that the amino residues which cause higher mitogenic signaling in the insulin molecule are ThrA⁸→His; GlnB⁴→His ; HisB¹⁰→Glu; ThrB²⁷→His. The possible reduction of mitogenic effect in insulin molecule containing “the higher mitogenic signaling residues” could be achieved throughout the following replacement:

AlaB¹⁴→Thr; LeuB¹⁷→Ser; PheB²⁵→Tyr-NH₂ and the missing B26-B30 amino residues of native human insulin.

The mitogenic curves of insulin, Radoyka I and Radoyka II insulin analogues showed a bell-shaped dose response (FIG. 13). It can be seen that B14 Ala→Thr, the member of hexamer forming surface, decreased mitogenic effect of Radoyka II compared with Radoyka I insulin analogue.

This could be a step forward for designing new insulin analogues with less toxic effects, especially when the need of combined properties are required.

It is known that both insulin and IGF-I can stimulate both mitogenic and metabolic effects. Insulin is considered as primarily metabolic and IGF-I as primarily a mitogenic effects. The controversial theory has been postulated by A. C. Moses et al., (1991, Press, Boca Raton, Ann Arbor, Boston, p. 245) whether insulin and IGF-I can undertake biological and metabolical signaling via their own receptors, or whether insulin exerts its mitogenic effects through its weak affinity binding to IGF-I receptor, and IGF-I its metabolic effects through the insulin receptor.

If LB cell line is washed with Hepes binding buffer to get rid of IGF-I receptors (according to the procedure in De Meyts' laboratory), in which case it is clear that insulin can induce both metabolic and mitogenic effects via its own receptor.

Since Radoyka I and Radoyka II insulin analogues on the insulin receptor displayed the same mitogenic effect as native insulin, it would be interesting to investigate and to compare their mitogenic signaling via IGF-I receptors.

The side-chains which make contact and determine specificity may be quite different between the two ligand-receptors systems.

Also, the Radoyka I and Radoyka II insulin analogues showed a lack of the disappearance of negative cooperativity above 0.1 μM. This is in accordance with some insulins mutated by J. Brange et al, (1990, Diabetes Care, 13:923) at the hexamer forming surface, i.e. residues A13 Leu or B17 Leu which continue to accelerate the dissociation rate of ¹²⁵I-A14 insulin at concentrations>0. μM. Moreover, the negative cooperativity of the insulin analogues decreased as their mitogenic potency increased (P. De Meyts, et al., (1993), Exp. Clin. Endocrinol. Leipzig, 101:102. In this, the Radoyka I and Radoyka II insulin analogues showed a concordance with the theory. The negative cooperativity of Radoyka I and Radoyka II is slightly lower than native insulin. This could explain the slightly increased thymidine incorporation observed in Radoyka I and Radoyka II insulin analogues.

### The Biological Assays were Done According to the Following References

De Meyts, P.:Bianco, A. R.; Roth, J. Site-site interactions among insulin receptors. Characterization of the negative cooperativity. (1976) J. Biol. Chem. 251:1877.

Heding, A.; De Meyts, P. Shymko, R. M. Correlation of biosensor-derived insulin analogue binding parameters with radioreceptor assays. Abstract, The Endocrine Society, (1994).

Smal, J.; Closset, J.; Hennen, G.; De Meyts, P.; Receptor binding properties and insulin-like effects of human growth hormone and its 110 kDa-variant in rat adipocytes, (1987) J. Biol. Chem., 262:11071.

Shymko, R. M.; Gonzales, N.; Backer, J. M.; White, M. F.; De Meyts, P. Binding kinetics of mutated insulin receptors in transfected cells grown in suspension culture. Application to the Tyr→Phe 960 insulin receptor mutant. (1989) Biochem. Biophys. Res. Commun. 164:191. All of which are incorporated herein by reference.

### EXAMPLE 6

Iodination of Analogue Designated Radoyka II Human Insulin Analogue

The main purpose of radioactive labeled insulin and insulin analogues as substrates is to be used at a physiological concentration in assays of degradation.

Reagents:

1. 0.06M Na₂HP0₄, pH 7.0 (all reagents diluted in this buffer)

2. 25 units/ml lactoperoxidase (Sigma L-2005, lot # 121H-3789)

3. 1:10,000 dilution of 30% H₂O₂

4. Radoyka II human insulin analogue

5. 1 mCi NaI

Procedure:200 μl of phosphate buffer were added to the lyophilized aliquot of Radoyka II human insulin analogue and mixed. 10 μl of lactoperoxidase solution and 1 mCi of NaI were added in that order. The reaction was initiated by the addition of 10 μl of hydrogen peroxide. The reagents were mixed and the reaction allowed to proceed at room temperature (approx. 22° C.) for 15 min. The reaction mix was then applied to an Econo-Pac P6 desalting column and eluted with phosphate buffer by gravity, and collected as ten 1 ml fractions. 250 μl of samples derived from fractions 5, 6, 7, and 8 were applied to HPLC column using an elution buffer of 0.2 M pH 4.0 ammonium acetate/acetonitrile system, isocratic and linear gradient described under “Mandic method”. Flow was maintained at 1.0 ml/min throughout the elution. Fractions (0.5 ml) of the elute were collected at 0.5 min intervals and counted directly in a Tracor Analytic γ-counter to determine the elution profile of radioactivity.

### EXAMPLE 7

### The Degradation of des-(B26-B30-[Glu-B¹⁰; Thr-B¹⁴; Ser-B¹⁷; Tyr-B²⁵—NH₂] Radoyka II Human Insulin Analogue by Insulin Degrading Enzyme (IDE)

### Assays of Insulin Degradation

### Trichloroacetic Acid (TCA) Assay

Insulin degradation was assayed by the loss of precipitability in 10% trichloroacetic acid. Preliminary studies were carried out to determine the dilution of enzyme which would yield 20% degradation of 125 I A14 mono-iodo porcine insulin. It was found that dilution of 1:30 gave 22.46% TCA solubility. TCA analysis of insulin degrading enzyme (IDE) was done with different solutions for 15 minutes of incubation time. The tubes were set up in triplicate according to the following protocol.

850 μl of 100 mM TRIS buffer pH 7.5

100 μl of ¹²⁵I A14 insulin

50 μl of IDE (dilutions:straight, 1:2, 1:5, 1:10, 1:20, 1:30, 1:40, 1:50, 1:100, blank

The reaction was terminated by the addition of 50 μl of 10% BSA (bovine serum albumin) and 250 μl of 50% trichloroacetic acid (TCA). The precipitate was removed by centrifugation for 15 min at 1750 g, and supernatant pellet was counted. The amount of degradation was calculated from the increase in TCA solubility of ¹²⁵I-A14 insulin in comparison with control incubation, t=0. In this type of insulin degradation assay 20% of ¹²⁵I-A14 soluble is required as standard condition.

### High Performance Liquid Chromatography (HPLC) Assay

Insulin degradation was also assessed by HPLC separations. They were performed using a Beckman model 332 liquid chromatography, equipped with a model 210 injector fitted with a 250 μl loop. The column used was a DuPont Zorbax C-8 (4.6 mm×250 mm; 6 μm particle diameter) maintained at 40° C. with a Bioanalytical System LC-22A temperature controller. The degradation products were eluted from the column with 0.2 M ammonium phosphate (pH 4)/acetonitile solvent system.

| | |
|---|---|
| Solvent A: | 85% 0.2 M pH 4 ammonium phosphate/15% acetonitrile |
| Solvent B: | 60% 0.2 M pH 4 ammonium phosphate/40% acetonitrile |

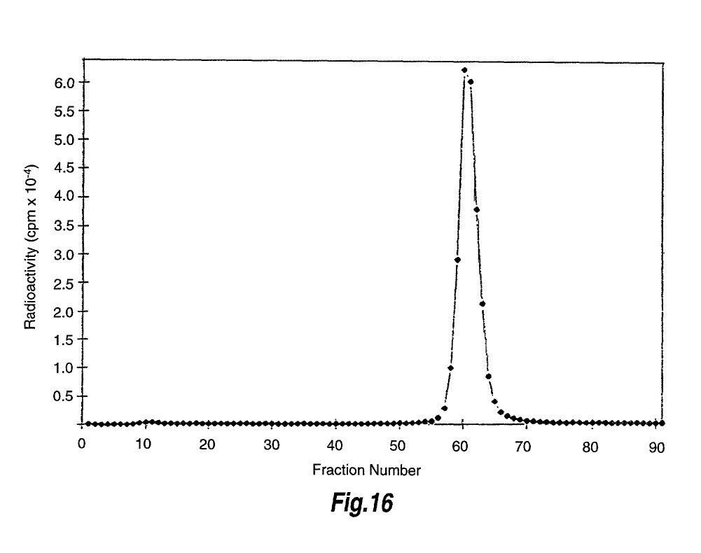

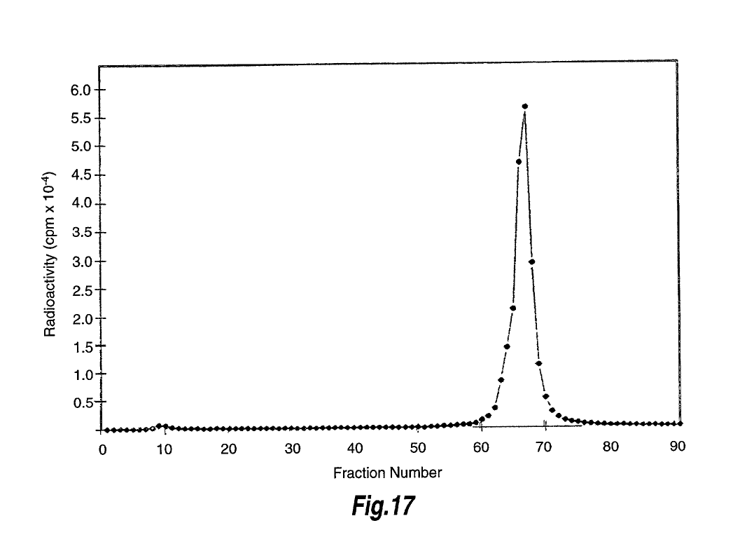

The isocratic and linear gradient steps used were described under “Mandic Method”. Flow was maintained at 1.0 ml/min throughout the elution. Fractions (0.5 ml) of the elute were collected at 0.5 min intervals. All degradation reactions of 125 A14 porcine insulin and the radiolabeled isomers of the Radoyka II human insulin analogue (collected under the eluents of the peaks P3; P5 shown in FIGS. 16; 17) were performed in bovine serum albumin (BSA) coated test tubes.

In order to obtain 20% TCA solubility of ¹²⁵I A14 insulin, preliminary experiments were performed at different times and dilutions of enzyme. These experiments showed that the best conditions were achieved when 25 μl of ¹²⁵I A14 insulin diluted with 480 μl of 100 mM TRIS buffer pH 7.5, 10 μl of insulin degrading enzyme (dilution 1:5, incubation times were 10 minutes and 20 minutes), and 465 μl of 100 mM TRIS buffer pH 7.5. The reaction mixture was subjected to TCA assay (Table 1) and HPLC assay. The HPLC elution profiles of degradation of ¹²⁵ I A14 in insulin and the radioactive isomers of Radoyka II human insulin are shown in FIGS. 18, 19, 20, 21, 22 and 23). The degradation of Radoyka II human insulin analogue was examined by TCA precipitability and by HPLC analysis and compared with insulin. The analogue was iodinated using lactoperoxidase and the monoiodinated isomers separated on HPLC, yielding three peaks termed P3, P4 and P5. If analogous to insulin, these would be the analogues labeled on the A19, B16 and A14 residues, respectively. All of the analogues isomers had reduced susceptibility to the degradation as compared with native insulin shown in Table 1.

### SUMMARY OF THE DESCRIPTION

Interest in insulin analogues is predicated upon the clinical need to improve upon existing commercial insulin preparations as well as the scientific need to understand fundamental mechanisms of the hormone.

The feasibility of preparing the human insulin analogues of this invention, as illustrated by the Radoyka I and Radoyka II human insulin analogues possessing enhanced stability towards insulin degrading enzyme (IDE) has been described and documented. The insulin degrading enzyme is the primary mechanism for insulin metabolism in the cell. The sites of cleavage of insulin by IDE have been established, demonstrating a limited number of cleavage sites by this enzyme. The goal, therefore, was developing and studying the properties of monomeric insulins with reduced susceptibility to degradation.

The novel human insulin analogues were synthesized by the peptide solid phase method:-Des-(B26-B30)-Glu-B¹⁰; Ser-B¹⁷; Tyr-B²⁵—NH₂], code name Radoyka I human insulin analogue; and -Des-(B26-B30)-[Glu-B¹⁰; Thr-B¹⁴; Ser-B¹⁰;Tyr-B²⁵—NH₂], code name Radoyka II human insulin analogue.

In the foregoing discussion, emphasis was directed to the positions at B14 and B17 as the primary sites of the protection from hydrolysis by IDE. The amino residues B10 and B25 are involved both in receptor binding and the sites of insulin cleavage by IDE. They were primarily considered in direction of increased affinity towards insulin receptor in order to make insulin analogues of the invention with a portion of insulin potency. The degradation of the insulin analogues of the invention have been shown 4.5 times less than that of porcine insulin.

This property can be of therapeutic importance in greater bioavailability of subcutaneously injected insulin and prolongation of its biological effect. It can lead to successful managing of the patient who has resistance to subcutaneous insulin, due to increased activity of IDE, a patient who has Mendenhall's syndrome, and those who have some renal disorders, etc.

Another desirable characteristic of the insulin analogues of the invention is for the molecules to remain in the monomer form rather than forming hexamers at higher concentrations.

A number of monomeric insulins have been developed and tested in humans, demonstrating more rapid absorption from subcutaneous tissue and more physiological insulin and glucose profiles after meals, as compared with regular insulin therapy.

Although several of these monomeric forms of insulin have been withdrawn from testing because of fears of enhanced mitogenicity or growth promotion, it is stressed that as the Radoyka I and Radoyka II human insulin analogues of the present invention have demonstrated in the biological assays the same mitogenicity as does native insulin.

It is contemplated that the insulin analogues of the invention have great potential as therapeutic agents for the treatment of human diabetes.

Since certain changes may be made without departing from the invention herein contemplated, it is intended that the description of the invention, including the specific examples, should be taken as illustrative only and not in a limiting sense. The scope of the invention should therefore be taken from the appended claims.

What is claimed is:

1. A human insulin analogue comprising the A-chain of human insulin and a mutant B-chain wherein the segment B26-B30 has been eliminated, further characterized by at least one of the following substitutions in the B-chain:
(1) replacing the B25 phenylalanine with a polar amino acid containing amine substitution of carboxylic acid groups or unsubstituted carboxylic acid groups adapted to increase resistance of the analogue to insulin-degrading enzyme;
(2) substitution at B14 of an amino acid that destabilizes the human insulin molecule towards formation of multimers and further increases resistance of the molecule to insulin-degrading enzyme;
(3) substitution at B17 of an amino acid that destabilizes the human insulin molecule towards formation of multimers and further increases resistance of the molecule to insulin-degrading enzyme;
(4) substitution at B10 of an amino acid that increases resistance to cleavage by insulin-degrading enzyme and alters insulin binding kinetics to insulin receptors in the body.

2. A pharmaceutical composition comprising the insulin analogue according to claim 1 and a pharmaceutically acceptable carrier.

3. A method for treating a patient having diabetes which comprises administrating to the patient a homeostasis-promoting amount of the insulin analogue of claim 1.

4. A method as defined in claim 3 wherein the homeostasis is maintained for at least 24 hours.

5. A method for treating a patient having subcutaneous insulin resistance which comprises administering to the patient a homeostasis-promoting amount of the insulin analogue of claim 1.

6. A method as defined in claim 5 wherein the homeostasis is maintained for at least 24 hours.

7. A method for treating a patient having Mendenhall's syndrome which comprises administering to the patient a homeostasis-promoting amount of the insulin analogue of claim 1.

8. A method as defined in claim 7 wherein the homeostasis is maintained for at least 24 hours.

9. A human insulin analogue as defined in claim 1 wherein said at least one substitution is at B25 wherein a polar amino acid containing amine substitution of carboxylic acid groups or unsubstituted groups adapted to increase resistance of the analogue to insulin-degrading enzyme is substituted for the phenylalanine substituent at B25 of the human insulin molecule.

10. A human insulin analogue as defined in claim 9 wherein the polar amino acid is selected from the group consisting of Thr, Ser, Gly, Cys, Tyr, Asn, Gln, Asp, Glu, Lys, Arg, His, OH-Pro and OH-Lys.

11. A human insulin analogue as defined in claim 1 wherein said at least one substituent is at B14 or B17 wherein an amino acid that destabilizes the insulin molecule towards formation of multimers is substituted for the phenylalanine substituent at B14 or the Leu substituent at B17, respectively, of the human insulin molecule.

12. A human insulin molecule as defined in claim 11 wherein the amino acid that destabilizes the insulin molecule towards formation of dimers, tetramers and lexamers is selected from the group consisting of Thr, Ser, Gly, Cys, Tyr, Asn, Gin, Asp, Glu, Lys, Arg, His, OH-Pro and OH-Lys.

13. A human insulin analogue as defined in claim 1 wherein said at least one substituent is at B14 and B1 7 wherein an amino acid that destabillizes the insulin molecule towards formation of multimers is substituted for the native substituent of the human insulin molecule.

14. A human insulin analogue as defined in claim 13 wherein the amino acid that desstabilizes the insulin molecule towards formation of multimers is selected from the group consisting of The, Ser, Gly, Cys, Tyr, Asn, Gln, Asp, Glu, Lys, Arg, His, OH-Pro and OH-Lys.

15. A human insulin analogue as defined in Clam 1 wherein said at least one substitution is at B10 wherein an amino acid that increases resistance to cleavage by IDE and alters insulin binding kinetics to insulin receptors in the body is substituted for the His of the human insulin molecule.

16. A human insulin analogue as defined in claim 15 wherein the at least one substituent is selected from the group consisting of Glu, Gln and Asn.
